(19) United States
(12) Patent Application Publication
(10) Pub. No.: US 2006/0211047 A1
(43) Pub. Date: Sep. 21, 2006

(54) Test kit

(75) Inventors: Minami Mutsuhiko, Yokohama City (JP); Hiroko Yamazaki, Morgan Hill, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: KineTech Diagnostics, Inc., Morgan Hill, CA (US)

(21) Appl. No.: 11/304,800

(22) Filed: Dec. 14, 2005

Related U.S. Application Data
(60) Provisional application No. 60/635,481, filed on Dec. 14, 2004.

(51) Int. Cl. G01N 33/53; G01N 33/00

(52) U.S. Cl. 435/7.1; 436/86

(57) ABSTRACT

It is an object of the present invention to significantly improve the treatment efficiency of bronchial asthma by intervening with inhaled steroids or the like and discontinuing the intervention at the most appropriate times respectively on the basis of accurately and objectively grasping the pathologic condition of an asthma patient among others. To achieve the above object, the present invention provides a method for inspecting the condition of a disease derived from a test sample by measuring the amounts of cytokines (IL-5, IL-6, IL-8, IL-10, TNF-α, IL-1β, IFN-γ, etc.) and/or chemokines in the test sample, and also a test kit for inspecting the condition of the disease, the test kit comprising a checking means for measuring the amounts of the cytokines in the test sample based on the method, a means for placing the test sample, and a means for allowing the test sample to access the checking means.

### CROSS-REFERENCE TO RELATED APPLICATION

The instant nonprovisional patent application claims priority to U.S. Nonprovisional Patent Application No. 60/635,481, filed Dec. 14, 2004 and incorporated by reference herein for all purposes.

### BACKGROUND OF THE INVENTION

1. Field of the Invention

The present invention relates to a test kit and a method for inspecting a disease by measuring the amounts of cytokines and/or chemokines in a sample of a patient's sputum, nasal mucus, serum, cerebrospinal fluid, or the like.

2. Background

Bronchial asthma is a disease wherein dyspnea is spasmodically repeated due to chronic inflammation of bronchial tubes and it is known that eosinophils play an important role. Patients with bronchial asthma that is one of modern diseases widely range from infants to adults and have recently been increasing in number. There exist approximately 1.5 million patients in Japan, of which 6,000 patients die due to an asthma attack every year.

There is still no curative therapy for the bronchial asthma, and therefore the pathologic condition is controlled by drug therapy as a symptomatic treatment. In particular, inhaled steroids based on atomization are widely used because they are very effective for improving the pathologic condition. However, it is known that the steroids cause various adverse effects, and therefore the use of the steroids should be minimized. In terms of the use of the steroids, if an asthma attack can be accurately predicted, using the steroids (intervention) before an asthma crisis would enable to prevent the attack.

Even if once the inhaled steroids were used, accurately evaluating a patient's pathologic condition to discontinue the use of steroids as early as possible would lead to the accurate control of asthma. However, neither accurately predicting a bronchial asthma attack nor accurately evaluating the pathologic stage of a patient suffering from the attack is easy under the present situation. References 1 to 6 listed at the end hereof describe the background art with respect to the above description.

In other words, if the pathologic condition can be accurately and objectively grasped, both intervening with inhaled steroids and discontinuing the intervention at the most appropriate times respectively would be possible, resulting in the significant improvement of the treatment of bronchial asthma.

However, in actual clinical examinations, the pathologic condition is evaluated only by clinical symptoms, peak flow rates (ventilatory capacity), and the like. Only with these types of information, accurately diagnosing the pathologic condition of bronchial asthma is not easy, and therefore the development of a method capable of more objectively and easily diagnosing the pathologic condition is desired. In other words, the establishment of a diagnostic method for the treatment of bronchial asthma is important, wherein the method can be employed as indicators for an earlier treatment intervention and the discontinuation of the intervention.

An attempt to determine a therapeutic strategy for an adult bronchial asthma patient has actually been made, based on evaluating the pathologic condition of the patient by carrying out a bronchoalveolar lavage for the patient followed by the examination of cell fractions and the like in the lavage fluid. However, the bronchoalveolar lavage is significantly invasive to a patient. On the other hand, obtaining induced sputum from a bronchial asthma patient is relatively easy and less invasive.

Recently, an attempt to control an asthma attack by using eosinophils in sputum of an infantile bronchial asthma patient as an indicator has been reported and resulted in the decrease in the number of patients who progressed to a critical condition or required hospitalization. The reference 6 listed at the end hereof discloses a method for inspecting a bronchial asthma attack based on the amount of E-cadherin decomposition products in a test sample (non-sputum sample).

In consideration of the above problems, the present invention relates to a method for grasping an asthma condition by measuring the changes in the amounts of cytokines and/or chemokines, based on the finding that the changes in the amounts of specific cytokines and/or chemokines are involved in the prediction or the termination of an asthma attack, and also to a kit for diagnosing the crisis and condition of asthma, wherein antibodies to the cytokines and chemokines are incorporated in the kit.

The present invention proposes specifically a method for inspecting the condition of a disease derived from a test sample by measuring the amounts of cytokines and/or chemokines in the test sample obtained from an asthma patient, and also a test kit for inspecting the condition of the disease, comprising a checking means for measuring the amounts of the cytokines in the test sample based on the method, a means for placing the test sample, and a means for allowing the test sample to access the checking means.

Thus, the present invention enables the pathologic condition of an asthma patient among others to be accurately and objectively grasped, whereby an intervention with inhaled steroids or the like and the discontinuation of the intervention can be implemented at the most appropriate times respectively, resulting in the provision of a method and a kit for significantly improving the treatment efficiency of bronchial asthma.

To achieve the above object, the present invention provides a method for inspecting the condition of a disease derived from a test sample by measuring the amounts of cytokines (IL-5, IL-6, IL-8, IL-10, TNF-α, IL-1β, IFN-γ, etc.) and/or chemokines in the test sample obtained from an asthma patient, and also a test kit for inspecting the condition of the disease, the test kit comprising a checking means for measuring the amounts of the cytokines in the test sample based on the method, a means for placing the test sample, and a means for allowing the test sample to access the checking means.

That is, the present invention predicts an asthma attack if the amount of IFN-γ increases in addition of the increase in the amounts of IL-5 and TNF-α, all of which are good indicators for predicting an asthma attack.

The present invention also predicts the termination of an asthma attack if the amounts of IL-5, IL-8 and IL-10 decrease, all of which are good indicators for determining the termination of an asthma attack.

### BRIEF SUMMARY OF THE INVENTION

An exemplary invention according to the present invention to solve the above-described problems is a test kit for inspecting the condition of a disease, the test kit comprising a sample-placing part and a detecting part for detecting a cytokine in a sample, wherein sputum is placed in the sample-placing part as a test sample, the detecting part detects an increase or a decrease in a concentration of the cytokine in the sample, and measuring the detected concentration of the cytokine enables to determine when an asthma attack is initiated or terminated.

The test kit may be characterized in that the cytokine to be detected in the detecting part includes any or all of IL-5, IL-6, IL-8, IL-10, TNF-α, IL-1β, and IFN-γ.

The test kit for determining when an asthma attack is initiated may be characterized in that the cytokine to be detected in the detecting part includes IL-5, TNF-α, and IFN-γ.

The test kit for determining when an asthma attack is terminated may be characterized in that the cytokine to be detected in the detecting part includes IL-5, IL-8, and IL-10.

A test kit for determining when an asthma attack is initiated wherein the cytokine to be detected in the detecting part includes IL-5, TNF-α, and IFN-γ and a test kit for determining when an asthma attack is terminated wherein the cytokine to be detected in the detecting part includes IL-5, IL-8, and IL-10 may be characterized by being configured as a physically single body.

The present invention enables the treatment efficiency of bronchial asthma to be significantly improved.

Therefore, the present invention can be used for a method and a kit for significantly improving the treatment efficiency of bronchial asthma.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a distribution graph of the amount of TNF-α detected in sputum obtained during a bronchial asthma attack as well as a non-attack.

FIG. 2 is a distribution graph of the amount of IL-5 detected in sputum obtained during a bronchial asthma attack as well as a non-attack.

FIG. 3 is a distribution graph of the amount of IL-6 detected in sputum taken during a bronchial asthma attack as well as a non-attack.

FIG. 4 is a distribution graph of the amount of IL-8 detected in sputum taken during a bronchial asthma attack as well as a non-attack.

FIG. 5 is a distribution graph of the amount of IL-10 detected in sputum taken during a bronchial asthma attack as well as a non-attack.

FIG. 6 is a distribution graph of the amount of IL-12 detected in sputum taken during a bronchial asthma attack as well as a non-attack.

FIG. 7 is a distribution graph of the amount of IL-1β detected in sputum taken during a bronchial asthma attack as well as a non-attack.

FIG. 8 is a distribution graph of the amount of IFN-γ detected in sputum taken during a bronchial asthma attack as well as a non-attack.

FIG. 9 is a graph illustrating the time courses of the changes in the concentrations of IL-5, IL-6, IL-8, and IL-10 in sputum after an attack in case 1.

FIG. 10 is a graph illustrating the time courses of the changes in the concentrations of IL-5, IL-6, IL-8, and IL-10 in sputum after an attack in case 2.

FIG. 11 is a schematic diagram illustrating a test kit for predicting an asthma attack, the test kit comprising a test sample, a control and monoclonal antibodies to cytokines; and a test kit for determining the termination of an attack, the test kit comprising of a test sample, a control and monoclonal antibodies to cytokines.

### DETAILED DESCRIPTION OF THE INVENTION

The present invention will hereinafter be described. The inventors have confirmed that the amounts of IL-1β and IFN-γ in sputum increased during an asthma attack as well as a non-attack; cytokines that increased specifically during an asthma attack but not increased during non-attack were TNF-α, IL-10 and IL-6; the amounts of IL-5, TNF-α, and IFN-γ in sputum increased during a non-attack in the case that an attack symptom appeared after the non-attack; and the concentration of the IFN-γ in the sputum increased even under a disease control during a non-attack after which an attack did not appear soon, and more increased immediately before an attack.

That is, the inventors have found that the concentrations of TNF-α, IL-5, IL-6, IL-8, and IL-10 in sputum increase as a bronchial asthma attack appears and therefore the increase and decrease in these cytokine concentrations may be able to be indicators for predicting the crisis and termination of asthma attack. Specific examples will be described below in reference to respective distribution graphs.

In addition, in the examples, a method for isolating cells in induced sputum of an infantile asthma patient was considered and by closely inspecting cell fractions of the induced sputum, an analysis on cells involved in the symptoms and pathologic condition of asthma was implemented. Against this background, a method for determining the condition of asthma was developed as well as the availability of the method was verified by easily and quickly measuring cytokines and the like in the induced sputum.

In the examples, sputum of an infantile asthma patient was fractionated into fractions of cellular and non-cellular elements to analyze the cell fractions as well as measure the concentrations of cytokines and chemokines in the non-cellular fractions, of which the relationship to the condition of asthma was investigated. Induced sputa were obtained from patients in various pathologic conditions to measure the concentrations of TNF-α, IL-2, IL-4, IL-5, IL-6, IL-8, IL-10, IL-12, IL-1β, and IFN-γ in the sputa.

Patients tested for bronchial hypersensitivity were selected from ambulatory or hospitalized patients due to bronchial asthma as objects of the investigation. Three different tests, i.e., inhalation thresholds (PC20) of acetylcholine and histamine, and an exercise-induced test, were performed for the bronchial hypersensitivity. From 35 bronchial asthma patients, 35 samples were obtained during a non-attack of asthma, 19 samples during an attack, and 10 samples immediately before an attack. Also, 7 healthy children who had no past history of allergic diseases and 9 children who was healthy but had infection of upper respiratory tract due to viruses or bacteria were selected as a control group and a case control group respectively.

Sputum was obtained after blowing his/her nose, gargling and spray inhalation of 4.5% normal saline solution with an ultrasonic nebulizer and then filtered centrifugally after adding to the sputum the same amount of 0.1% dithiothreitol as the sputum followed by a 15-minute treatment.

For the measurements of cytokines and chemokines, the concentrations of TNF-α, IL-2, IL-4, IL-5, IL-6, IL-8, IL-10, IL-12, IL-1β, and IFN-γ in the respective sputa were measured with a CBA (Cytometric Bead Array) manufactured by Becton Dickinson.

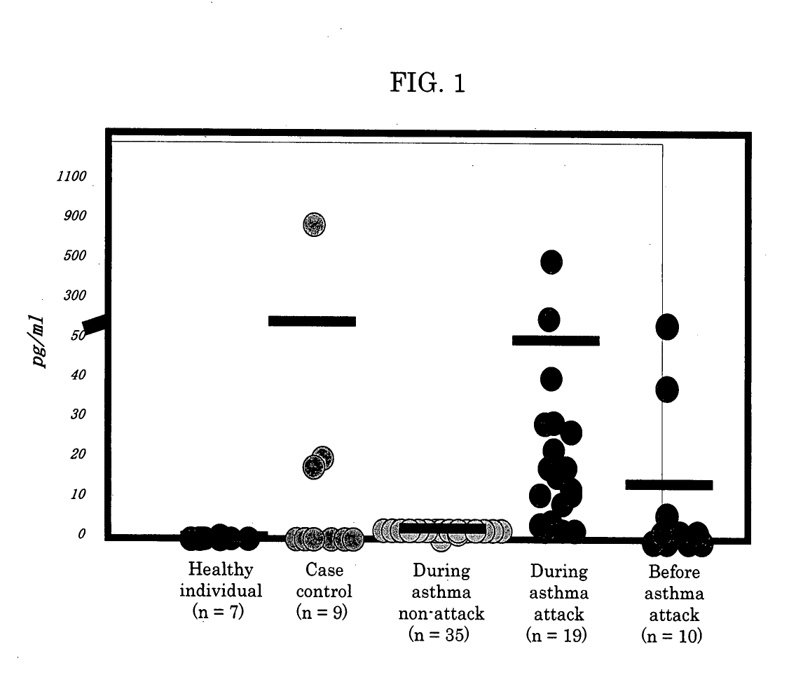

The results of the measurements are shown in FIGS. 1 to 10. FIG. 1 is a distribution graph of the amount of TNF-α detected in sputum obtained during a bronchial asthma attack as well as a non-attack. As shown in the graph, the concentration of TNF-α increases during an asthma attack in many cases, and also increases in some cases of infection of upper respiratory tract. Also as shown in FIG. 1, TNF-α is not detected in sputa of healthy children at all, while the concentration of it increases in 3 samples out of the 9 in the case-control group (infection of upper respiratory tract). On the other hand, in the asthma non-attack group (35 samples), TNF-α is not detected in any samples, while in the asthma attack group, the concentration of TNF-α increases in 13 samples out of the 19. That is, the concentration of TNF-α increases during an asthma attack in many cases, and also increases in some cases of infection of upper respiratory tract.

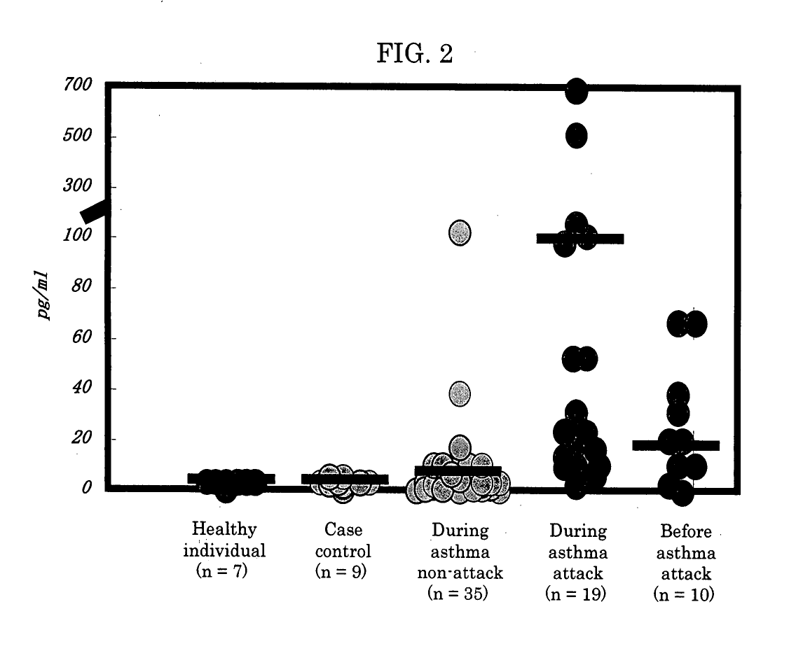

FIG. 2 is a distribution graph of the amount of IL-5 detected in sputum obtained during a bronchial asthma attack as well as a non-attack. As shown in the graph, it can be said that the concentration of IL-5 increases characteristically during an asthma attack.

Specifically, as shown in FIG. 2, the concentration of IL-5 does not increase for the healthy children and the case-control group. On the other hand, the concentration of IL-5 increases only in 2 samples out of the 35 in the asthma non-attack group, while it increases in 15 samples out of the 19 in the attack group. That is, it can be said that the concentration of IL-5 increases characteristically during an asthma attack. However, it should be noted that the concentration of IL-5 increases in a few cases even in the non-attack group.

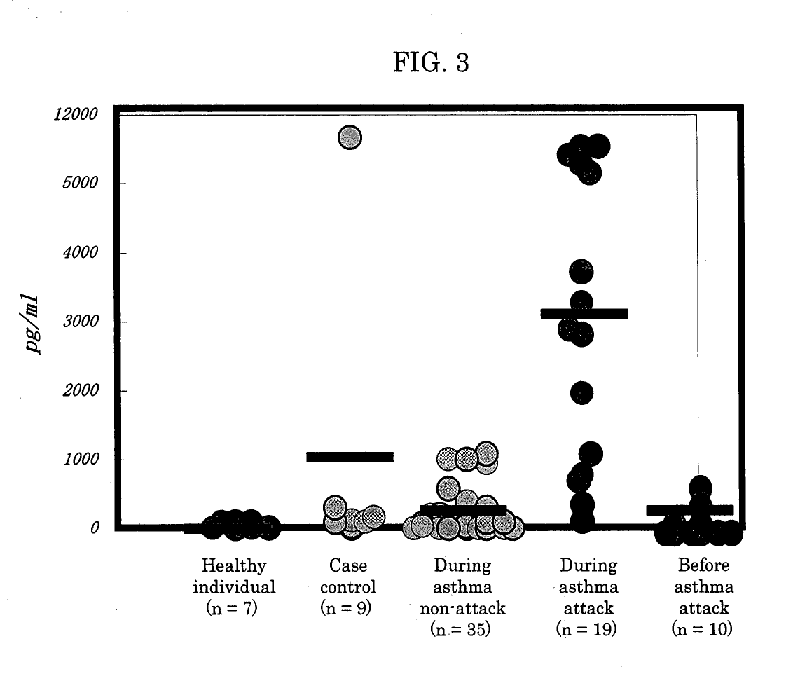

FIG. 3 is a distribution graph of the amount of IL-6 detected in sputum obtained during a bronchial asthma attack as well as a non-attack. As shown in the graph, it can be said that the concentration of IL-6 increases characteristically during an asthma attack.

Specifically, as shown in FIG. 3, the concentration of IL-6 does not increase for the healthy children and increases only in 1 sample in the case-control group. On the other hand, the concentration of IL-6 increases only in 5 samples out of the 35 in the asthma non-attack group, while it increases in 17 samples out of the 19 in the attack group. That is, it can be said that the concentration of IL-6 increases characteristically during an asthma attack. However, it should be noted that the concentration of IL-6 increases in a few cases even in the non-attack group.

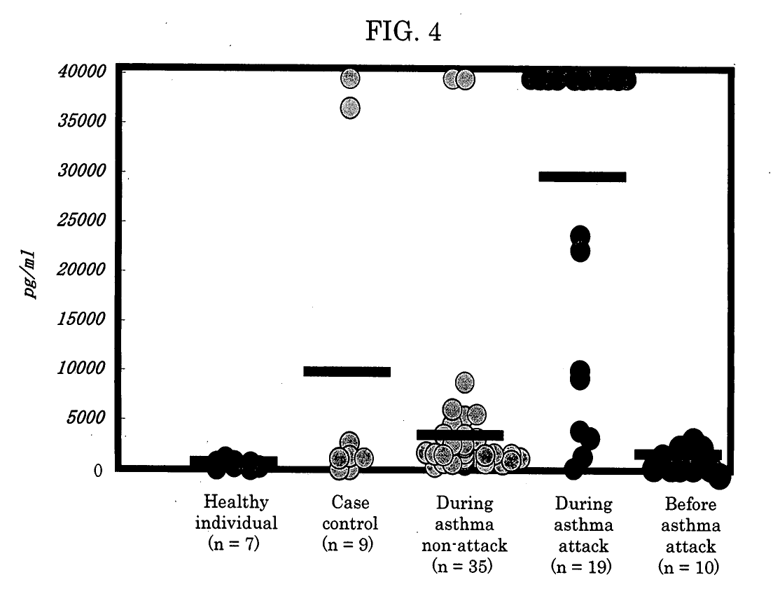

FIG. 4 is a distribution graph of the amount of IL-8 detected in sputum obtained during a bronchial asthma attack as well as a non-attack. As shown in the graph, it can be said that the concentration of IL-8 increases characteristically during an asthma attack.

Specifically, as shown in FIG. 4, the concentration of IL-8 does not increase for the healthy children and increases only in 2 samples in the case-control group. On the other hand, the concentration of IL-8 increases only in 6 samples out of the 35 in the asthma non-attack group, while it increases in 15 samples out of the 19 in the attack group. That is, it can be said that the concentration of IL-8 increases characteristically during an asthma attack. However, it should be noted that the concentration of IL-8 increases in a few cases even in the non-attack group.

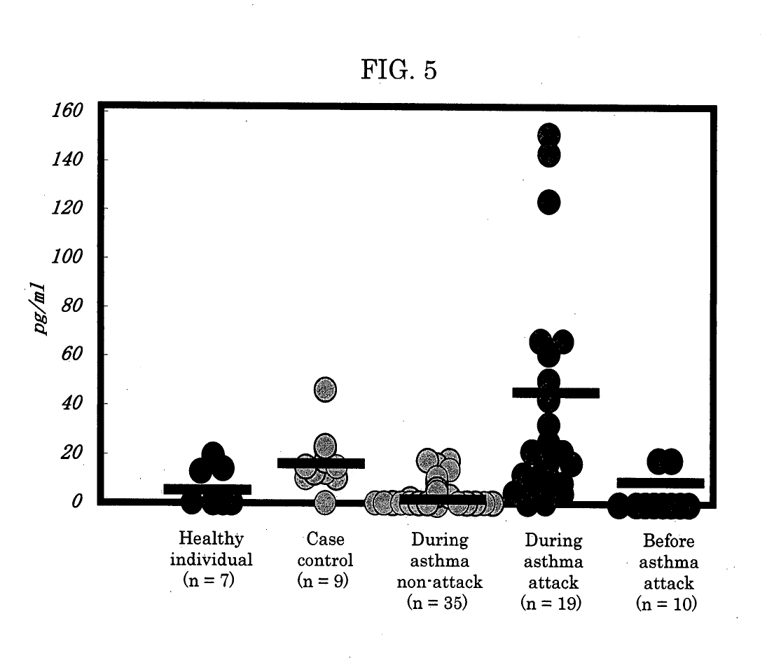

FIG. 5 is a distribution graph of the amount of IL-10 detected in sputum obtained during a bronchial asthma attack as well as a non-attack. It can be said that the concentration of IL-10 increases during an asthma attack, which may be considered to be in contrast with the non-attack group in which the increase in the IL-10 concentration cannot be seen.

Specifically, as shown in FIG. 5, the concentration of IL-10 does not increase at all for the healthy children and slightly increases in 1 sample in the case-control group. On the other hand, the concentration of IL-10 does not increase in the asthma non-attack group, while it increases in 8 samples out of the 19 in the attack group. That is, it can be said that the concentration of IL-10 increases during an asthma attack, which may be considered to be in contrast with the non-attack group in which the increase in the IL-10 concentration cannot be seen.

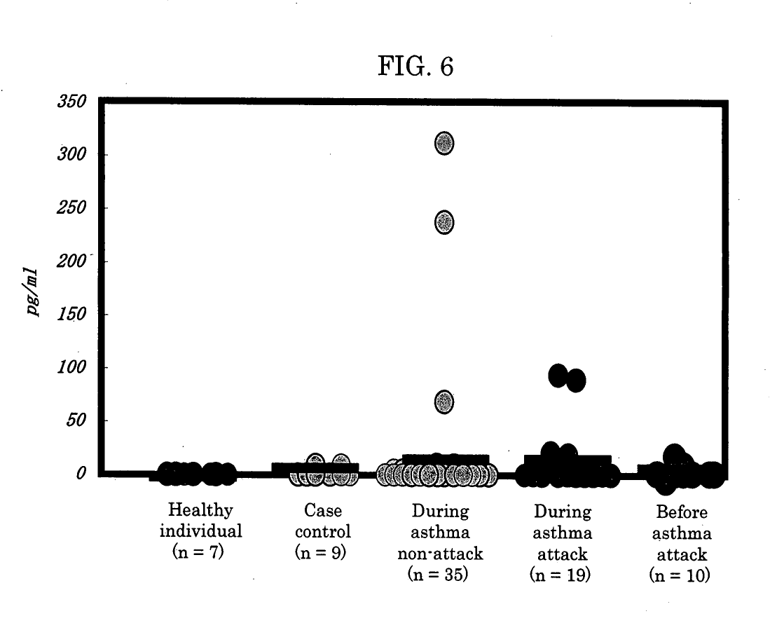

FIG. 6 is a distribution graph of the amount of IL-12 detected in sputum obtained during a bronchial asthma attack as well as a non-attack. As shown in the graph, the concentration of IL-12 slightly increases in 2 samples out of the 19 in the attack group.

Specifically, as shown in FIG. 6, the concentration of IL-12 does not increase at all for the healthy children and the case-control group. On the other hand, the concentration of IL-10 increases in 3 samples out of the 35 in the asthma non-attack group and also slightly increases in 2 samples out of the 19 in the attack group.

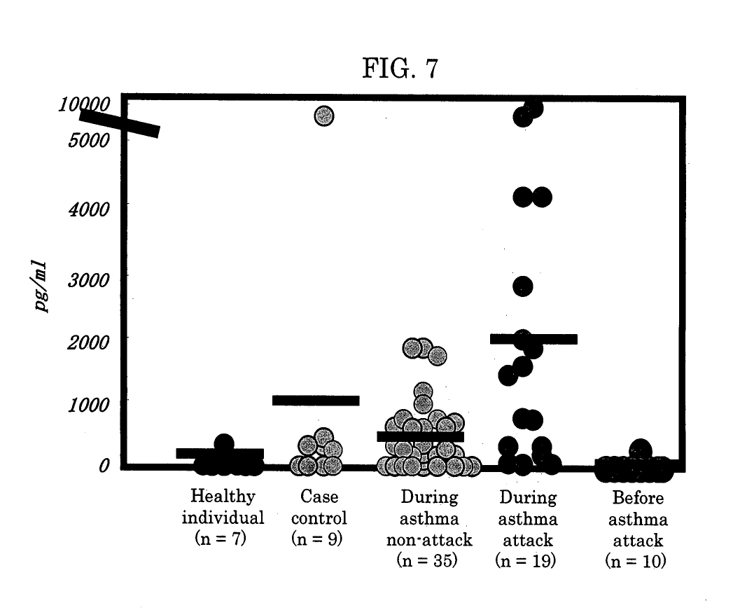

FIG. 7 is a distribution graph of the amount of IL-1β detected in sputum obtained during a bronchial asthma attack as well as a non-attack. As shown in the graph, it can be thought that the concentration of IL-1β increases during an asthma attack and also slightly increases in the non-attack group.

Specifically, as shown in FIG. 7, the concentration of IL-1β does not increase at all for the healthy children and increases only in 1 sample in the case-control group. On the other hand, the concentration of IL-10 slightly increases in 5 samples out of the 35 in the asthma non-attack group and also increases in 9 samples out of the 19 in the attack group. That is, it can be thought that the concentration of IL-1β increases during an asthma attack and also slightly increases even in the non-attack group.

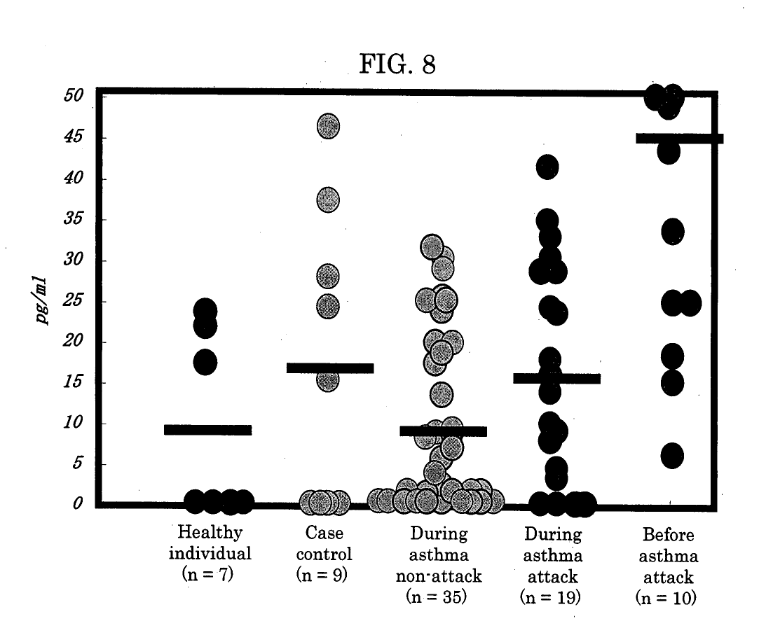

FIG. 8 is a distribution graph of the amount of IFN-γ detected in sputum obtained during a bronchial asthma attack as well as a non-attack. As shown in the graph, it can be seen that the concentration of IFN-γ increases during an inflammation including an asthma attack.

Specifically, as shown in FIG. 8, the concentration of IFN-γ increases in 3 samples out of the 7 even for the healthy children and in 5 samples out of the 9 of the case-control group. On the other hand, the concentration increases in 12 samples out of the 35 of the asthma non-attack group and in 12 samples out of the 19 of the attack group. Also, the concentration significantly increases in some cases immediately before an asthma attack. That is, it can be thought that the concentration of IFN-γ increases during an inflammation including an asthma attack.

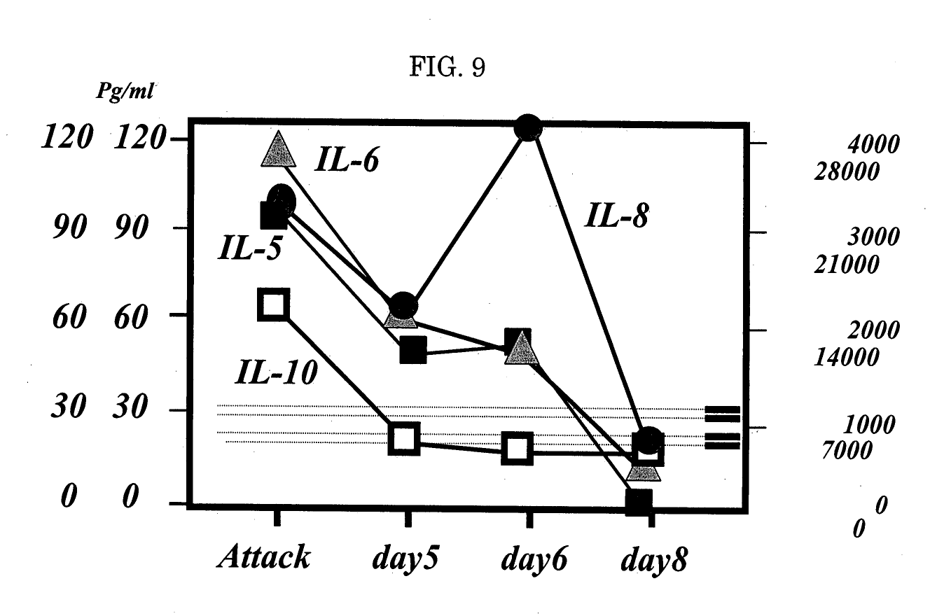

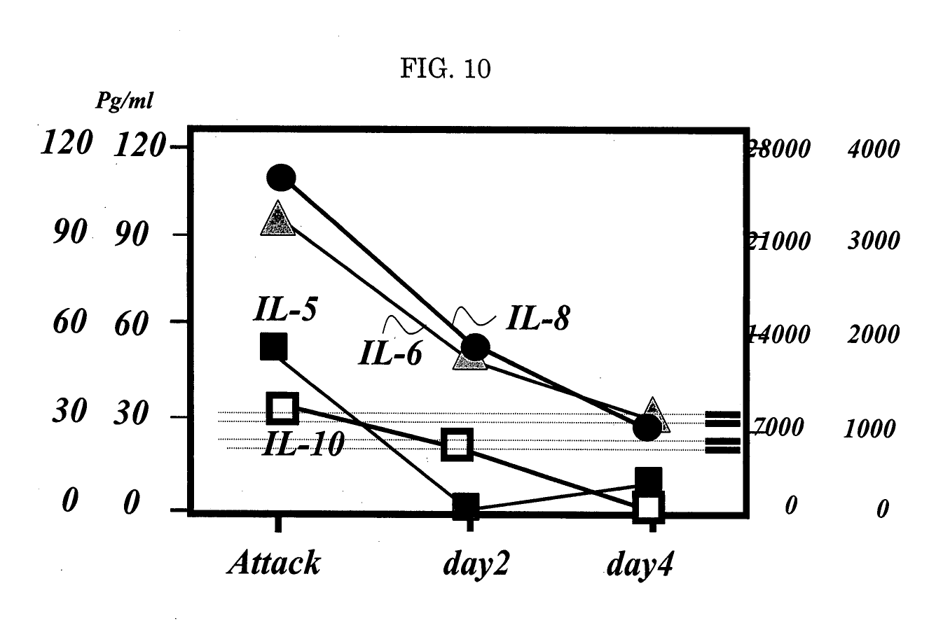

FIG. 9 is a graph illustrating the time courses of the changes in the concentrations of IL-5, IL-6, IL-8, and IL-10 in sputum after an attack in case 1 and FIG. 10 a graph illustrating the time courses of the changes in the concentrations of IL-5, IL-6, IL-8, and IL-10 in sputum after an attack in case 2.

The time courses of the changes in the concentrations of IL-5, IL-6, IL-8, and IL-10 in sputum after an attack were investigated in connection with an asthma attack by measuring with time the concentrations of cytokines in sputa in two cases where pathological conditions of two infantile bronchial asthma patients could be observed with time after an attack (FIGS. 9 and 10). In the case 1, as shown in FIG. 9, among 4 cytokines, i.e., IL-5, IL-6, IL-8, and IL-10, of which concentrations increased during an asthma attack, the concentration of IL-10 decreases first followed by the decrease in the concentrations of IL-5 and IL-6, and then the concentration of IL-8 decreases.

On the other hand, for the time courses of the changes in the concentrations of IL-5, IL-6, IL-8, and IL-10 in sputum after an attack in the case 2, as shown in FIG. 10, among IL-5, IL-6, IL-8, and IL-10 of which concentrations increased during an asthma attack, the concentration of IL-10 decreases first followed by the decrease in the concentration of IL-5, and then the concentrations of IL-6 and IL-8 decrease almost simultaneously.

From the above measurement results, it is found that before an asthma attack the concentration of IFN-γ in sputum tends to exhibit the highest value and that of IL-5 the second highest value. It is also found that during an asthma attack the concentration of IL-5 in sputum increases as well as those of IL-8 and IL-10 and after the attack the concentrations of IL-5, IL-8 and IL-10 decrease with time and exhibit lower values. The changes in the concentrations of these cytokines can be considered to be superior indicators of an earlier intervention for a good prognosis and of the completion of treatment. Thus, developing a method for easily examining cytokines as well as eosinophils in sputum enables a patient's pathologic condition to be quickly and more accurately evaluated at the site of medical care.

One embodiment of the present invention is a method for inspecting the condition of a disease, comprising the step of detecting the amounts of cytokines and/or chemokines in a test sample. Another embodiment may be a method for inspecting the condition of a disease, comprising the step of detecting the amounts of cytokines and/or chemokines in a sputum, nasal mucus, serum, or cerebrospinal fluid sample.

Still another embodiment may be a method for inspecting the condition of a disease, comprising the step of detecting the amount of cytokines in a patient's sputum during a bronchial asthma attack (including an infantile asthma attack) as well as a non-attack. Still another embodiment may be a method for inspecting the condition of a disease, comprising the step of detecting TNF-α, IL-5, IL-6, IL-8, IL-10, IL-12, IFN-γ, or IL-1β in a patient's sputum with its corresponding monoclonal antibody, i.e., anti-TNF-α, anti-IL-5, anti-IL-6, anti-IL-8, anti-IL-10, anti-IL-12, anti-IFN-γ, or anti-IL-1β during a bronchial asthma attack as well as a non-attack.

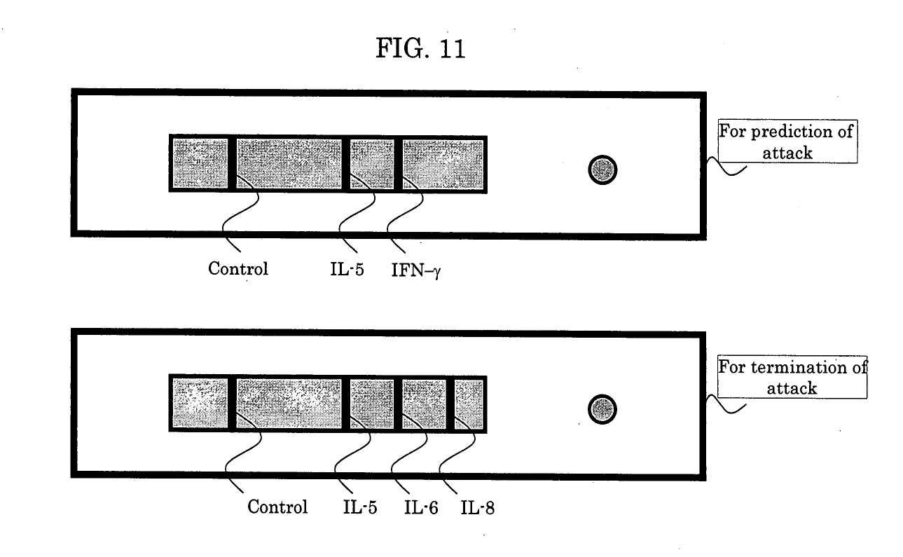

FIG. 11 is a schematic diagram illustrating a test kit for predicting an asthma attack, comprising a test sample, a control and monoclonal antibodies to cytokines (three types, i.e., anti-IL-5, anti-TNF-α, and anti-IFN-γ monoclonal antibodies); and a test kit for determining the termination of an asthma attack, comprising a test sample, a control and monoclonal antibodies to cytokines (three types, i.e., anti-IL-5, anti-IL-8 and anti-IL-10 monoclonal antibodies).

In addition, a specific example of a test kit according to the present invention includes a kit comprising anti-IL-5, anti-IL-8 and anti-IL-10 monoclonal antibodies; a control; and a test sample, as the test kit for predicting an asthma attack shown in FIG. 11. On the other hand, the termination of an asthma attack can be predicted on the basis of the decrease in the concentration of IL-10 and determined to be completely terminated on the basis of the decrease in the concentrations of IL-5, IL-6 and IL-10.

A specific example of a kit comprises anti-IL-5, anti-IL-8 and anti-IL-10 monoclonal antibodies; a control; and a test sample, as the test kit for determining the termination of an asthma attack shown in FIG. 11.

A determination whether or not an intervention should be implemented can be made in such a way that the intervention is initiated if the increase in the concentrations of IL-5 and TNF-α (IFN-γ) in sputum is observed. Also, a determination whether or not an already-initiated intervention should be discontinued can be made in such a way that the termination of an attack is first predicted on the basis of the decrease in the concentration of IL-10 in sputum and then the intervention can be discontinued if the decrease in the concentrations of IL-5, (IL-6), and IL-8 is found.

This enables a pathologic condition to be easily and quickly grasped at an early stage, whereby the prevention of an asthma attack and a determination of a therapeutic strategy based on a long-range perspective become possible. In particular, with the use of serum, sputum, or nasal mucus obtained by a noninvasive sampling approach, measuring the variations in the concentrations of eosinophils cationic protein (ECP) and various cytokines that are indicators of eosinophilic inflammation enables the condition of a bronchial asthma to be effectively diagnosed.

In addition, one embodiment of the present invention discloses that a test kit for predicting an attack and that for determining the termination of an attack are separated from each other; however, without being limited to this, a single test kit on one plate may comprise a common test sample for both predicting an attack and determining the termination of an attack.

An inspection method and a test kit according to the present invention enable the pathologic condition of a patient to be accurately and objectively grasped by timely detecting the amounts of cytokines and/or chemokines in sputum obtained from the patient when treating bronchial asthma, whereby an intervention with inhaled steroids or the like and the discontinuation of the intervention can be implemented at the most appropriate times respectively, resulting in the effect of significantly improving the treatment efficiency of bronchial asthma.

Another embodiment is a test kit for inspecting the condition of a disease, the test kit comprising a checking means for measuring the amounts of the cytokines in a test sample; a means for placing the test sample; and a means for allowing the test sample to access the checking means. Still another embodiment is a test kit for inspecting the condition of a disease, the test kit comprising a checking means for detecting a cytokine in sputum with its corresponding monoclonal antibody.

Still another embodiment is a test kit for inspecting the condition of a disease, the test kit comprising a checking means having at least two, preferably three, monoclonal antibodies, which are preliminarily disposed in a line and selected from the group consisting of anti-TNF-α, anti-IFN-γ, anti-IL-5, anti-IL-8, and anti-IL-10 that are monoclonal antibodies to TNF-α, IFN-γ, IL-5, IL-8, and IL-10 respectively.

Still another embodiment may be a test kit for predicting a bronchial asthma attack, the test kit comprising a checking means having anti-TNF-α, anti-IL-5 and anti-IFN-γ monoclonal antibodies preliminarily disposed in a line. Still another embodiment may be a test kit for predicting a bronchial asthma attack, the test kit comprising a checking means having anti-TNF-α and anti-IL-5 monoclonal antibodies preliminarily disposed in a line. In addition, preferably, the concentrations of cytokines to be detected in test samples of the above kits are 30 pg/ml or more for TNF-α, 20 pg/ml or more for IL-5, and 20 pg/ml or more for IFN-γ.

Still another embodiment may be a test kit for determining the termination of a bronchial asthma attack, the test kit comprising a checking means having anti-IL-5, anti-IL-8 and anti-IL10 monoclonal antibodies preliminarily disposed in a line. Still another embodiment may be a test kit for determining the termination of a bronchial asthma attack, the test kit comprising a checking means having anti-IL-5 and anti-IL-8 monoclonal antibodies preliminarily disposed in a line. In addition, preferably, the concentrations of cytokines to be detected in test samples of the above kits are 30 pg/ml or more for IL-10, 20 pg/ml or more for IL-5, 1000 pg/ml or more for IL-6, and 6000 pg/ml or more for IL-8.

Still another embodiment may be a test kit for predicting a bronchial asthma attack or a test kit for determining the termination of a bronchial asthma attack, wherein a test sample is allowed to access a monoclonal antibody to a cytokine on the basis of the principle of chromatography. In order to visually express the presence of various cytokines with their corresponding concentration ranges described above, any color former commonly employed in an immunochromatography method can be used, and gold colloid is especially preferred in the present invention.

An inspection method and a test kit according to the present invention are not limited to the above-described embodiments, and similar inspection methods and test kids are within the technical scope of the present invention without departing the object and effects of the present invention. For example, a test kit for predicting an attack and that for determining the termination of an attack are physically separated from each other in FIG. 11; however, without being limited to this, inspections for both predicting an attack and determining the termination of an attack may be implemented with a single test kit.

The following references are incorporated by reference herein for all purposes: Tockman et al., “Safe separation of sputum cells from mucoid glycoprotein”, Acta Cytol., 39, 1128-1136 (1995); Simpson et al., “Optimization of sputum-processing methods for measurement of interleukin-5: effects of protease inhibition”, Respirology, 7-111-116 (2002); Dominguez Ortega et al., “Fluorcytometric analysis of induced sputum cells in an asthmatic population”, J. Investig. Allergol. Clin. Immunol., 14, 108-113 (2004); Beier, J. et al., “Induced sputum methodology: Validity and reproducibility of total glutathione measurement in supernatant of healthy and asthmatic individuals”, J. Lab. Clin. Med., 144, 38-44 (2004); and PCT published application no. WO/01/053832.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A test kit for inspecting a condition of a disease, the test kit comprising a sample-placing part and a detecting part for detecting a cytokine in a sample, wherein sputum is placed in the sample-placing part as a test sample;
the detecting part detects an increase or a decrease in a concentration of the cytokine; and
measuring the detected concentration of the cytokine enables to determine when an asthma attack is initiated or terminated.

2. The test kit according to claim 1, wherein the cytokine to be detected in the detecting part includes any or all of IL-5, IL-6, IL-8, IL-10, TNF-α, IL-1β, and IFN-γ.

3. The test kit according to claim 2, the test kit for determining when an asthma attack is initiated, wherein the cytokine to be detected in the detecting part includes IL-5, TNF-α, and IFN-γ.

4. The test kit according to claim 2, the test kit for determining when an asthma attack is terminated, wherein the cytokine to be detected in the detecting part includes IL-5, IL-8, and IL-10.

5. The test kit according to claim 2, wherein said test kit for determining when an asthma attack is initiated in which the cytokine to be detected in the detecting part includes IL-5, TNF-α, and IFN-γ and said test kit for determining when an asthma attack is terminated in which the cytokine to be detected in the detecting part includes IL-5, IL-8, and IL-10 are configured as a physically single body.
